(19) United States
(12) Patent Application Publication
(10) Pub. No.: US 2022/0175570 A1
(43) Pub. Date: Jun. 9, 2022

(54) Posture Correcting Apparatus

(71) Applicant: BHM IP Pty. Ltd., Carseldine (AU)

(72) Inventor: Khaled Akbery, Carseldine (AU)

(73) Assignee: BHM IP Pty. Ltd., Carseldine (AU)

(21) Appl. No.: 17/437,338

(22) PCT Filed: Mar. 6, 2020
(86) PCT No.: PCT/AU2020/050209

§ 371 Date: Sep. 8, 2021

(30) Foreign Application Priority Data
Mar. 8, 2019 (AU) 2019900776

(51) Int. Cl. A61F 5/02

(52) U.S. Cl. CPC A61F 5/026

(57) ABSTRACT

A postural training and correction apparatus to assist a user of the apparatus to recognize and correct faulty posture, the apparatus comprising: a substantially rigid rod assembly extending between an in use top end and an in-use bottom end; a head engagement member positioned and attached to at the top end of the rod assembly for receiving and engaging the user's head to prompt the user to position the head in engagement with the top end of the rod member; a shoulder strapping arrangement being coupled to the rod assembly for being positioned in between the top and bottom ends of the rod assembly comprising a pair of resilient shoulder loops for extending around the user's left and right shoulders respectively, and a waist or hip engagement member attached to the bottom end of the rod assembly for engaging the user's abdomen or hip; wherein during use the rigid rod assembly is adapted to be substantially positioned along the spine of the user to contact the user's body at least three separate locations thereby indicating to the user that the user has attained a correct posture.

### TECHNICAL FIELD

The present invention relates to a posture correcting apparatus for recognising and correcting posture of a user.

### BACKGROUND

Any references to methods, apparatus or documents of the prior art are not to be taken as constituting any evidence or admission that they formed, or form part of the common general knowledge.

A common characteristic of many different types of otherwise unrelated activities is the necessity of good posture in order to perform them successfully and without undue risk of injury. In particular, appropriate spinal posture (in layman's terms, a “straight spine”) is critical to ensuring a good result and preventing back injury.

One activity of particular interest is the act of using free weights for exercising in a gymnasium. One of the most difficult activities to master in order to achieve good results and avoid injury is the maintenance of appropriate spinal posture. Maintaining a correct posture usually requires a person to align their pelvis, abdomen, shoulders and head.

One of the most common ways in which chiropractors and other sports and medical professionals instruct patients to stand against a wall with the head, pelvis and shoulders touching the wall whilst they carry out an activity (such as performing squats). However, such a technique places several limitations on the patient and there is at least a need to provide an improved alternative for assisting people with improving and maintaining correct posture.

### SUMMARY OF INVENTION

In one aspect, the invention provides a postural training and correction apparatus to assist a user of the apparatus to recognize and correct faulty posture, the apparatus comprising:

a substantially rigid rod assembly extending between an in use top end and an in-use bottom end;

a head engagement member positioned and attached to the top end of the rod assembly for receiving and engaging the user's head to prompt the user to position the head in engagement with the top end of the rod member;

a shoulder strapping arrangement being coupled to the rod assembly for being positioned in between the top and bottom ends of the rod assembly comprising a pair of resilient shoulder loops for extending around the user's left and right shoulders respectively, and

a waist or hip engagement member attached to the bottom end of the rod assembly for engaging the user's abdomen or hip;

wherein during use the rigid rod assembly is adapted to be substantially positioned along the spine of the user to contact the user's body in at least three separate locations thereby indicating to the user that the user has attained a correct posture.

In an embodiment, the rod assembly comprises: a central section for contacting the back of the user with an in-use top section having said top end and an in-use bottom section having said bottom end.

In an embodiment, each of the top section and the bottom section is movably relative to the central section.

In an embodiment, each of said top section and bottom section is telescopically received at opposite ends of the central section.

In an embodiment, the central section comprises a flattened profile with at least one flat surface for engagement with the user's back.

In an embodiment, the head engagement member comprises a profiled head contacting projection provided on the top section of the rod assembly.

In an embodiment, the waist or hip engagement member comprises a profiled waist or hip contacting projection provided on the bottom section of the rod assembly.

In an embodiment, the in use length between the top and bottom ends of the rod assembly is variable.

In an embodiment, the resilient shoulder straps apply a resilient bias to apply a pulling force on the shoulder blades of the user to attain the correct posture.

In an embodiment, the shoulder strapping arrangement is adapted to be attached in between ends of the central section at a plurality of attachment locations.

In an embodiment, the shoulder strapping arrangement comprises a back panel flanked by said shoulder loops with each of the shoulder loops extending from respective upper and lower side portions of the back panel.

In an embodiment, the apparatus further comprises a flexible sleeve located on the back panel for receiving the rod assembly therein such that during use when the shoulder loops are disposed over the shoulders of the user, the sleeve is located to position the received rod assembly to be substantially in alignment with at least a portion of the user's spine.

In an embodiment, the postural training and correction apparatus further comprises a fastener to secure the flexible sleeve between inner walls of the fastener and outer peripheral portions of the central section.

In an embodiment, the fastener comprises a snap on fastener with resiliently biased portions to grip the flexible sleeve and apply a biasing force to secure the flexible sleeve between inner walls of the fastener and outer peripheral portions of the central section.

In an embodiment, the peripheral portions on the central section are provided along a relatively narrow region of the central section to receive the biased portions of the snap on fastener with relatively broader regions being located at the ends of the central section.

In another aspect, the invention provides a method of recognising and correcting posture for a user of a postural training and correction apparatus wherein the apparatus comprises: a substantially rigid rod assembly extending between an in use top end and an in-use bottom end; a head engagement member positioned and attached to the top end of the rod assembly for receiving and engaging the user's head to prompt the user to position the head in engagement with the top end of the rod member; a shoulder strapping arrangement being coupled to the rod assembly for being positioned in between the top and bottom ends of the rod assembly comprising a pair of resilient shoulder loops for extending around the user's left and right shoulders respectively, and a waist or hip engagement member attached to the bottom end of the rod assembly for engaging the user's abdomen or hip; the method comprising the steps of:

extending each shoulder loop of the shoulder strapping arrangement around the user's left and right shoulders respectively to position the rod assembly of the apparatus relative to the user's back such that the top end is positioned adjacent the user's head and the bottom end is positioned adjacent to the user's pelvis;

positioning the head engagement member adjacent the user's head and pushing the user's head to engage the head engagement member; and

positioning the waist or hip engagement member to engage the user's pelvis such that during use the rigid rod assembly is adapted to be substantially positioned along the spine of the user to contact the user's body at least three separate locations thereby indicating to the user that the user has attained a correct posture.

### BRIEF DESCRIPTION OF THE DRAWINGS

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way. The Detailed Description will make reference to a number of drawings as follows:

FIG. 1 is a frontal view of a postural training and correction apparatus 100 in accordance with an embodiment of the present invention.

FIG. 2 is a rear view of the postural training and correction apparatus 100.

FIG. 3 is side view of the postural training and correction apparatus 100.

FIG. 4 is a left hand side perspective view of the postural training and correction apparatus 100.

FIG. 5 is a right hand side perspective view of the postural training and correction apparatus 100.

FIG. 6 is an isolated frontal view of the shoulder strap assembly 140 which forms part of the postural training and correction apparatus 100.

FIG. 7 is a side view of the shoulder strap assembly 140.

FIG. 8 is a rear view of the shoulder strap assembly 140.

FIG. 9 is a left hand perspective view of the shoulder strap assembly 140.

FIG. 10 is a right hand perspective view of the shoulder strap assembly 140.

FIG. 11 is frontal view of the rod assembly 110 which forms part of the postural training and correction apparatus 100.

FIG. 12 is a side view of the rod assembly 110.

FIG. 13 is a rear view of the rod assembly 110.

FIG. 14 is left hand side perspective view of the rod assembly 110.

FIG. 15 is a right hand side perspective view of the rod assembly 110.

FIG. 16 is a left hand side perspective view (exploded) of the postural training and correction apparatus 100.

FIG. 17 is a right hand side perspective view (exploded) of the postural training and correction apparatus 100.

### DETAILED DESCRIPTION OF PREFERRED EMBODIMENTS

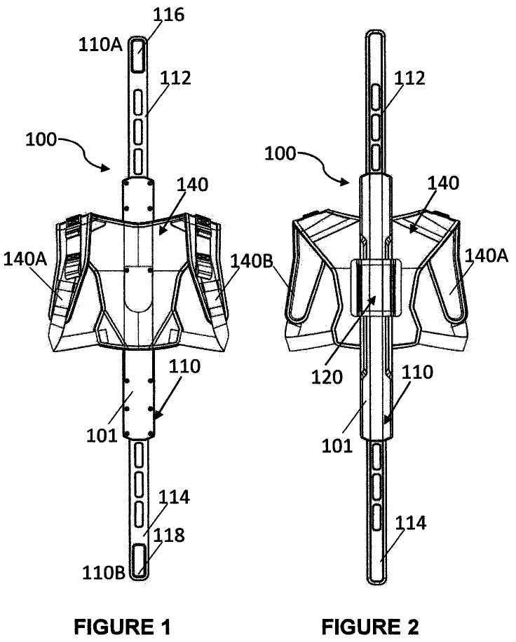

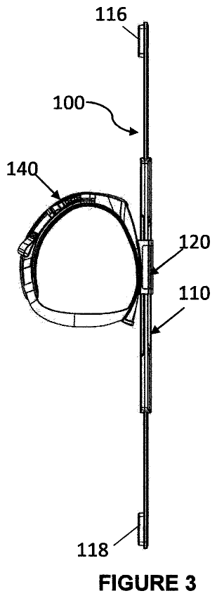

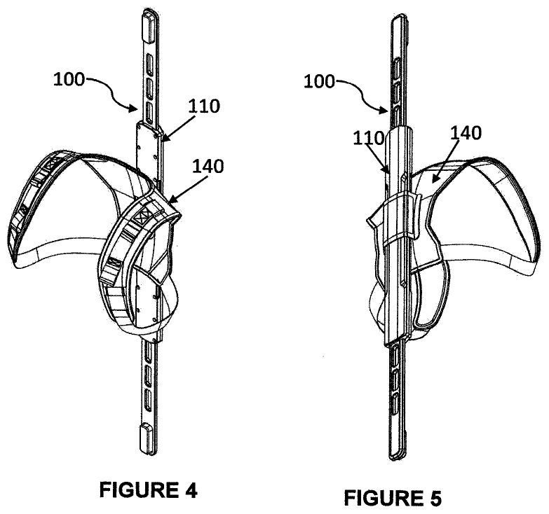

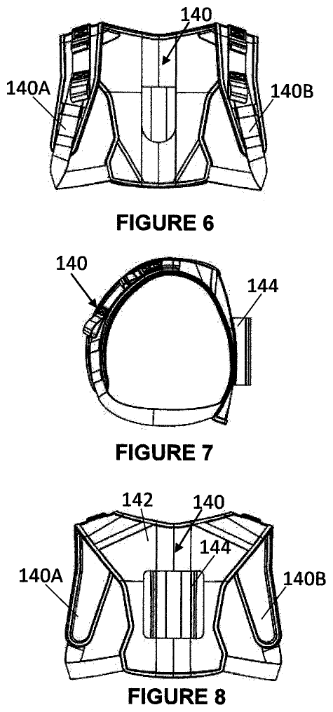

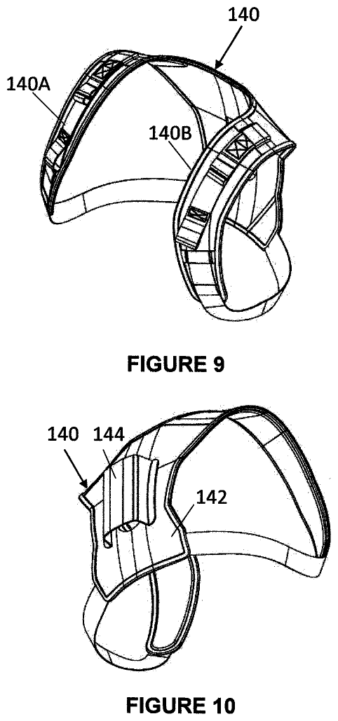

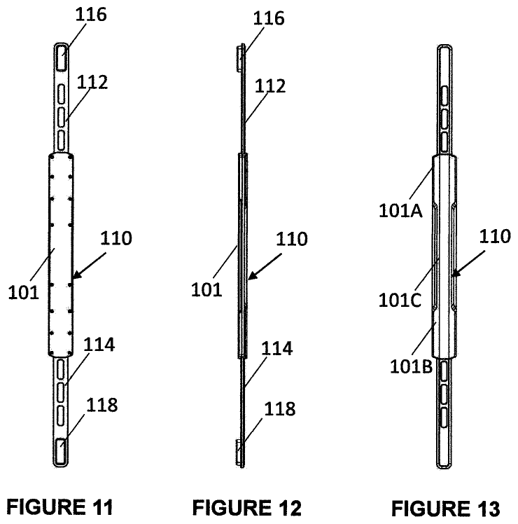

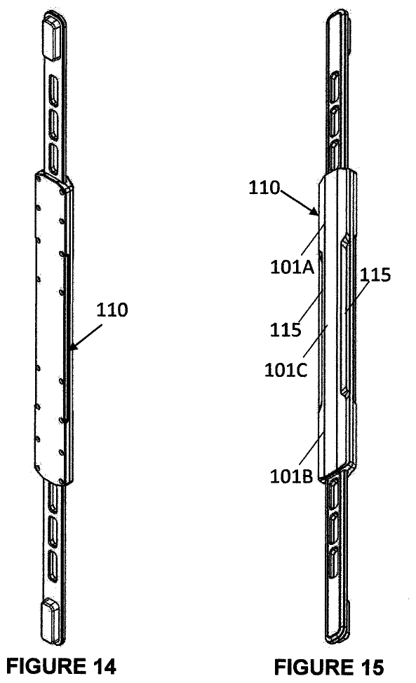

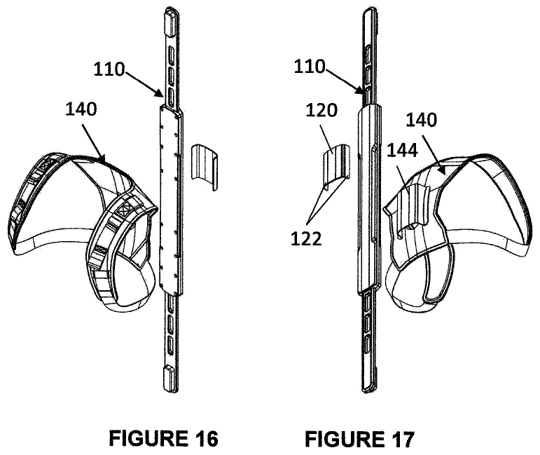

FIGS. 1 to 17 illustrate a postural training and correction apparatus 100 which is useful for assisting users of the apparatus 100 to recognize and correct faulty posture as will be explained in the foregoing sections.

The apparatus 100 comprises a substantially rigid rod assembly 110 that extends between an in use top end 110A and an in-use bottom end 110B. In the presently described embodiment, the rigid rod assembly 110 comprises a central section 101 with an in-use top section 112 and in-use bottom section 114 that are telescopically connected to the central section 101. In the preferred embodiment, the central section comprises a flattened and hollow profile that is elongate. The central section has two ends 101A and 101B (See FIG. 13) which receive the top section 112 and the bottom section 114 respectively. The telescopic arrangement of the rod assembly 110 allows the overall length of the rod assembly 110 to be varied. The telescopic connecting arrangement allows the top section 112 and the bottom section 114 to be connected in a plurality of telescopic positions thereby allowing the overall length of the rigid rod assembly 110 to be varied easily. Some of the advantages associated with allowing the overall length of the rod assembly 100 to be varied will be discussed in the foregoing sections. The central section 101, top section 112 and the bottom section 114 have an elongate and flattened profile. However, the shape of the central section 101 and the top and bottom sections 112 and 114 may vary. For example, in some embodiments the central section 101 and the top and bottom sections 112 and 114 may be cylindrical and constructed from carbon fibre, wood, metal, injection moulded polymer or any other substantially rigid material.

The top section 112 includes a profiled head engagement portion 116 that is located at an upper end of the top section 112. As discussed in previous sections, the in-use height of the top section 112 may be adjusted by telescope movement (upward or downward) relative to the central section 101 to ensure that during use, the user can move their head into a corrected posture by contacting the occipital region of the their head with the head engagement portion 116. Typically, an incorrect posture of the user comprises the user's head falling in a forwardly direction. The appropriate positioning of the head engagement portion 116 allows the user to move their head in a backward direction to touch the occipital region of their head against the head engagement portion 116 thereby receiving immediate feedback that an improved posture has been achieved. It is also important to distinguish contact at the external occipital protuberance from mere contact with the back of the head. If the individual is making contact with the head engagement portion 116 using their external occipital protuberance, spinal neutrality is ensured. However, if the individual is only to make contact to any part of the rod assembly 110 then spinal neutrality is less likely to be ensured. The provision of the profiled head engagement portion 116 and the pelvis engagement portion 118 prompts the user to make contact with the profiled portions of the rod assembly 110 and not just rely on contact with any of the other remaining portions of the top section 112 or the bottom section 114.

Similarly, the bottom section 114 comprises a profiled pelvis or hip engagement portion 118 that is located at a lower end of the bottom section 114. As discussed in previous sections, the in-use height of the bottom section 112 may be adjusted by telescope movement (upward or downward) relative to the central section 101 to ensure that during use, the user can move their pelvis into a corrected posture by contacting the medial sacral crest region of the user with the pelvis engagement portion 118. As previously discussed, an incorrect posture of the user comprises the user's head falling in a forwardly direction and the pelvis not being in line with the user's head. The appropriate positioning of the pelvis engagement portion 118 allows the user to move their pelvis to touch the medial sacral crest region of the user's pelvis against the pelvis engagement portion 118 thereby allowing the use to receive immediate feedback that an improved posture has been achieved.

A shoulder strapping arrangement 140 is also provided to secure and retain the rod assembly 100 relative to the user's body during use. The shoulder strapping arrangement 140 comprises a harness or a brace with two shoulder receiving loops 140A and 140B that are inter-connected to each other via a back panel 142. During use, the user can insert their left and right arms through each of the loops 140A and 140B such that the shoulder loops 140A and 140B extend around the user's left and right shoulders respectively. Once again the shoulder straps 140A and 140B are preferably formed from an elastic material to pull the user's shoulders back towards an in-use central portion of the rod assembly 110. The length of the loops 140A and 140B may also be easily adjustable. The back panel 142 is preferably formed from a flexible fabric material to allow the user to wear the shoulder strap assembly 140 like a harness system. A dorsal portion of the back panel 142 is provided with a flexible sleeve 144 that is suitably sized to receive the central section 101 of the rod assembly 110. The provision of the sleeve 144 prevents the rod assembly 110 from contacting the user's back directly. The location of the flexible sleeve 144 allows the attachment location of the shoulder strap assembly 140 to be varied which provides an important advantage.

The presently described apparatus 100 ensures a neutral spinal position by providing the rigid rod assembly 110 with an effectively linear and planar surface that makes contact with the dorsum of the user. The head engagement member 116, the back panel 142 (attached to the central section 101) and the pelvis engagement member 118 makes contact at the approximate levels of the external occipital protuberance, spinous processes of the mid-thoracic spine, and the median sacral crest respectively. If the user expresses too much cervical extension, contact may be lost between the rigid member surface and the mid-thoracic spine. However, the harness arrangement provided by the strap assembly 140 prevents the movement of the back panel 142 and the attached central section 101 of the rod assembly 110 thereby reducing the extent of cervical extension. If the individual expresses too much cervical flexion, contact may be lost between the head engagement member 116 and the external occipital protuberance of the user. The loss of feedback from the profiled head engagement member 116 would prompt the user to reduce the cervical flexion in order to maintain contact between the head engagement member 116 and the occipital region of the user. If the user's body undergoes excessive thoracic extension, the central section 100 of the rigid rod assembly 110 also moves resulting in the pelvis engagement section 118 pushing against the user's pelvis prompting the user to return to the neutral spine position. Similarly, if the user undergoes excessive lumbar extension, the central section 101 attached to the back panel 142 of the shoulder strap assembly 140 will undergo some movement which will result in the head engagement member 116 becoming spaced away from the user's occipital region and the pelvis engagement member 118 pushing onto the user's pelvis region thereby prompting the user to return to the neutral position. If the user undergoes excessive lumbar flexion, contact may be lost between the pelvis engagement member 118 and the user's median sacral crest. In addition, a pushing force would also be applied on the user's occipital region thereby prompting the user to return to the neutral position.

The provision of the profiled head engagement member 116 and the pelvis engagement members 118 allows the user to realise when contact with any other region of the top or bottom sections 112 and 114 and instantly allows the user to correct their posture to ensure that the occipital region and the medial sacral crest region of the user contact the correct portion of the rod assembly 110 thereby improving the chances of the user returning to the correct neutral posture.

The correct positioning of the head engagement member 116 and the pelvis engagement member 118 (by adjusting the in-use length of the top section 112 and the bottom section 114) is not entirely sufficient to achieve the desire outcomes. The position of the shoulder strap assembly 140, particularly the attachment location of the sleeve 144 along the length of the central section 101 is an important consideration. In the presently described embodiment, it is desirable to position the sleeve 144 (and the shoulder strap assembly 140) in a manner to ensure that the sleeve 144 is positioned adjacent vertebrae in the mid-thoracic region of the user. A snap on fastener 120 is provided to allow the attachment location of the sleeve 144 to be varied relative to the length of the central section 101 of the rod assembly 110. The snap on fastener 120 includes resiliently biased portions 122 (See FIG. 17) to grip the flexible sleeve 144 and apply a biasing force to secure the flexible sleeve 144 between inner walls of the fastener 120 and outer peripheral portions 115 of the central section 101 (shown in FIG. 15). The peripheral portions 115 on the central section 101 are provided along a relatively narrow region of the central section to receive the biased portions 122 of the snap on fastener 120 with relatively broader regions being located at the ends 101A and 101B of the central section 101. Such a configuration allows the attachment location of the shoulder strap assembly 140 to be varied and fixed at the appropriate location.

The apparatus 100 therefore allows the in-use location/position of the head engagement member 116, the pelvis engagement member 118 and the attachment location of the strap assembly 140 to be varied independently relative to each other thereby making the apparatus 100 more versatile.

In compliance with the statute, the invention has been described in language more or less specific to structural or methodical features. The term “comprises” and its variations, such as “comprising” and “comprised of” is used throughout in an inclusive sense and not to the exclusion of any additional features.

It is to be understood that the invention is not limited to specific features shown or described since the means herein described comprises preferred forms of putting the invention into effect.

The invention is, therefore, claimed in any of its forms or modifications within the proper scope of the appended claims appropriately interpreted by those skilled in the art.

What is claimed is:

1. A postural training and correction apparatus to assist a person wearing the apparatus to recognize and correct faulty posture, the apparatus comprising:
a substantially rigid rod assembly extending between a top section and a bottom section of the apparatus;
a profiled head engagement member positioned and attached to the top section of the rod assembly for receiving and engaging the person's head to prompt the person to position the person's head in engagement with the top end of the rod member;
a shoulder strapping arrangement being coupled to the rod assembly and positioned in between the top and bottom sections of the rod assembly comprising a pair of resilient shoulder loops for extending around a left and a right shoulder of the person, respectively, and
a profiled waist or hip engagement member attached to the bottom section of the rod assembly for engaging an abdomen or a hip of the person;
wherein the rigid rod assembly is adapted to be substantially positioned along a spine of the person to contact a body of the person in at least three separate locations thereby indicating to the person that the person has attained a correct posture; and
wherein the rod assembly comprises:
a central section with a hollow body for contacting a back of the person with a top section having a top end and a bottom section having a bottom end;
wherein each of the top section and the bottom section is movable relative to the central section; and
wherein each of said top section and bottom section is telescopically received into the hollow body of the central section at the top end and bottom end respectively; and
wherein the shoulder strapping arrangement comprises:
a back panel flanked by said shoulder loops with each of the shoulder loops extending from respective upper and lower side portions of the back panel;
a flexible sleeve located on the back panel for receiving the rod assembly therein such that when the shoulder loops are disposed over the shoulders of the person, the sleeve is located to position the received rod assembly to be substantially in alignment with at least a portion of the person's spine; and
a fastener to secure the flexible sleeve between inner walls of the fastener and outer peripheral portions of the central section.

2. A postural training and correction apparatus in accordance with claim 1 wherein the central section comprises a flattened profile with at least one flat surface for engagement with the person's body.

3. A postural training and correction apparatus in accordance with claim 1 wherein the head engagement member comprises a profiled head contacting projection provided on the top section of the rod assembly.

4. A postural training and correction apparatus in accordance with claim 1 wherein the waist or hip engagement member comprises a profiled waist or hip contacting projection provided on the bottom section of the rod assembly.

5. A postural training and correction apparatus in accordance with claim 1 wherein the length between the top and bottom ends of the rod assembly is variable.

6. A postural training and correction apparatus in accordance with claim 1 wherein the resilient shoulder straps apply a resilient bias to apply a pulling force on the shoulder blades of the person to attain the correct posture.

7. A postural training and correction apparatus in accordance with claim 1 wherein the shoulder strapping arrangement is adapted to be attached at a plurality of attachment locations in between ends of the central section at a plurality of attachment locations.

8. A postural training and correction apparatus in accordance with claim 1 wherein the flexible sleeve provides the only attachment mechanism for attachment of the shoulder strapping arrangement.

9. A postural training and correction apparatus in accordance with claim 1 wherein the fastener comprises a snap on fastener with resiliently biased portions to grip the flexible sleeve and apply a biasing force to secure the flexible sleeve between inner walls of the fastener and outer peripheral portions of the central section.

10. A postural training and correction apparatus in accordance with claim 9 wherein the peripheral portions on the central section are provided along a relatively narrow region of the central section to receive the biased portions of the snap on fastener with relatively broader regions being located at the ends of the central section.

11. A method of recognising and correcting a posture of a person wearing a postural training and correction apparatus wherein the apparatus comprises: a substantially rigid rod assembly extending between a top end and a bottom end of the apparatus;
a profiled head engagement member positioned and attached to the top end of the rod assembly for receiving and engaging the person's head to prompt the person to position the person's head in engagement with the top end of the rod member;
a shoulder strapping arrangement being coupled to the rod assembly and positioned in between the top and bottom ends of the rod assembly comprising a pair of resilient shoulder loops for extending around a left and a right shoulders of the person, respectively, and
a profiled waist or hip engagement member attached to the bottom end of the rod assembly for engaging an abdomen or a hip of the person;
wherein during use the rigid rod assembly is adapted to be substantially positioned along a spine of the person to contact a body of the person in at least three separate locations thereby indicating to the person that the person has attained a correct posture; and
wherein the rod assembly comprises:
a central section with a hollow body for contacting a back of the person with a top section having said top end and a bottom section having said bottom end;
wherein each of the top section and the bottom section is movable relative to the central section; and
wherein each of said top section and bottom section is telescopically received into the hollow body of the central section at the top end and bottom end respectively; and
wherein the shoulder strapping arrangement comprises:
a back panel flanked by said shoulder loops with each of the shoulder loops extending from respective upper and lower side portions of the back panel;
a flexible sleeve located on the back panel for receiving the rod assembly therein such that when the shoulder loops are disposed over the shoulders of the person, the sleeve is located to position the received rod assembly to be substantially in alignment with at least a portion of the person's spine; and
a fastener to secure the flexible sleeve between inner walls of the fastener and outer peripheral portions of the central section; and
the method comprising the steps of:
extending each shoulder loop of the shoulder strapping arrangement around the person's left and right shoulders respectively to position the rod assembly of the apparatus relative to the person's back and telescopically adjusting the top and bottom ends to align with the person's head and pelvis such that the top end is positioned adjacent to the person's head and the bottom end is positioned adjacent to the person's pelvis;
positioning the head engagement member adjacent the person's head and pushing the person's head to engage the head engagement member; and
positioning the waist or hip engagement member to engage the person's pelvis such that the rigid rod assembly is adapted to be substantially positioned along the spine of the person to contact the person's body in at least three separate locations thereby indicating to the user that the user has attained a correct posture.
